(19) United States
(12) Patent Application Publication
(10) Pub. No.: US 2023/0255827 A1
(43) Pub. Date: Aug. 17, 2023

(54) PIEZO-DRIVEN AGITATOR FOR THE TREATMENT OF GLAUCOMA AND METHODS OF USE

(71) Applicant: Clauson Creative Engineering, Reno, NV (US)

(72) Inventors: Luke W. Clauson, Reno, NV (US); Brendan C. Reese, Reno, NV (US)

(21) Appl. No.: 18/170,428

(22) Filed: Feb. 16, 2023

Related U.S. Application Data
(60) Provisional application No. 63/268,092, filed on Feb. 16, 2022.

(51) Int. Cl. A61F 9/007

(52) U.S. Cl. CPC A61F 9/00781; A61B 17/32002

(57) ABSTRACT

An intraocular device for treating glaucoma comprising a treatment probe comprising a distal end region and a proximal end region, the distal end region including a distal end effector sized to penetrate the trabecular meshwork; an outer sheath surrounding at least the proximal end region of the treatment probe; and a drive mechanism operatively coupled to a proximal end of the treatment probe and configured to cause oscillatory movement of the proximal end of the treatment probe to vibrate the distal end effector and agitate intraocular tissue in contact with the distal end effector. Related devices, systems, and methods are provided.

### CROSS-REFERENCE TO RELATED APPLICATIONS

This application claims the benefit of priority under 35 U.S.C. §119(e) to co-pending U.S. Provisional Pat. Application Serial No. 63/268,092, filed Feb. 16, 2022. The disclosure of the application is incorporated by reference in its entirety.

### BACKGROUND

Glaucoma is a complicated disease in which damage to the optic nerve leads to progressive vision loss and is the leading cause of irreversible blindness. Aqueous humor is the fluid that fills the anterior chamber in front of the iris and the posterior chamber of the eye behind the iris. Vitreous humor or vitreous body is a gel-like material found in the posterior segment of the eye posterior of the capsular bag. FIG. 1 is a diagram of the front portion of an eye 5 showing the lens 7, cornea 8, iris 9, ciliary body 6 including ciliary processes 4, trabecular meshwork 10, and Schlemm’s canal 12. The aqueous humor is a fluid produced by the ciliary body 6 that lies behind the iris 9 adjacent to the lens 7. This aqueous humor washes over the lens 7 and iris 9 and flows to the drainage system located in the angle of the anterior chamber 16. The angle of the anterior chamber 16, which extends circumferentially around the eye 5, contains structures that allow the aqueous humor to drain.

Some of the aqueous humor is absorbed through the trabecular meshwork 10 into Schlemm’s canal 12 into collector channels and passing through the sclera 15 into the episcleral venous circulation. The trabecular meshwork 10 extends circumferentially around the anterior chamber 16 in the angle. The trabecular meshwork 10 limits the outflow of aqueous humor. Schlemm’s canal 12 is located beyond the trabecular meshwork 10. The two arrows in the anterior chamber 16 of FIG. 1 show the flow of aqueous humor from the ciliary body 6, over the lens 7, over the iris 9, through the trabecular meshwork 10, and into Schlemm’s canal 12 and its collector channels.

In some cases glaucoma is caused by blockage of aqueous humor outflow such as by sclerosis of the trabecular meshwork, pigment or membrane in the angle. In other cases, blockage is due to a closure of the angle between the iris and the cornea. This angle type of glaucoma is referred to as “angle-closure glaucoma”. In the majority of glaucoma cases, however, called “open angle glaucoma”, the cause is unknown.

Treatments of glaucoma attempt to lower intraocular pressure (IOP) pharmacologically or by surgical intervention that enhance outflow of aqueous humor through the outflow pathways. Ab externo trabeculectomy is a type of glaucoma surgery that creates a new path as a “controlled” leak for fluid inside the eye to drain out. Conventionally, a partial thickness scleral flap is formed followed by the creation of a small hole into the anterior chamber. Aqueous humor can flow into the subconjunctival space creating a filtering bleb. The scleral flap is raised up and a blade used to enter the anterior chamber. During the operation a hole is created under the scleral flap that is fluidically connected to the anterior chamber creating an opening. The opening is partially covered with the scleral flap. A small conjunctival “bleb” or bubble appears over the scleral flap, often near the junction of the cornea and the sclera (limbus).

Minimally-invasive surgical procedures provide IOP lowering by enhancing the natural drainage pathways of the eye with minimal tissue disruption. Minimally-invasive glaucoma surgery (MIGS) uses microscopic-sized equipment and tiny incisions. MIGS offers an alternative to conventional glaucoma surgeries with the potential benefit of reducing a patient’s dependence on topical glaucoma medication. Trabeculectomies and trabeculotomies can each be performed ab interno, or from inside the anterior chamber. Ab interno approaches aim to decrease IOP by increasing aqueous humor outflow through a direct opening in the trabecular meshwork from within the anterior chamber so that there is direct communication between the anterior chamber and the outer wall of Schlemm’s canal. Ab interno approaches include the TRABECTOME (MST / NeoMedix Corp.) electrosurgical instrument that ablates and removes trabecular meshwork, the Kahook Dual Blade (New World Medical) for excisional goniotomy removing a strip of trabecular meshwork, gonioscopy assisted transluminal trabeculotomy (GATT) involving cutting through the trabecular meshwork, cannulating Schlemm’s canal, and Omni (Sight Sciences) for performing viscoplasty or trabeculotomy through an ab interno approach for cannulating Schlemm’s canal. Other ab interno methods include the iStent (Glaukos) to create pathway through the trabecular meshwork for improved outflow of aqueous humor through Schlemm’s canal.

In view of the foregoing, there is a need for improved devices and methods related to ophthalmic surgery for the treatment of glaucoma.

### SUMMARY

In an aspect, described an intraocular device for treating glaucoma including a treatment probe having a distal end region and a proximal end region, the distal end region including a distal end effector sized to penetrate the trabecular meshwork; an outer sheath surrounding at least the proximal end region of the treatment probe; and a drive mechanism operatively coupled to a proximal end of the treatment probe and configured to cause oscillatory movement of the proximal end of the treatment probe to vibrate the distal end effector and agitate intraocular tissue in contact with the distal end effector.

The drive mechanism can further include a piezoelectric crystal stack. The piezoelectric crystal stack can be driven at a frequency of 50 Hz - 40 kHz to vibrate the distal end effector. The distal end effector can vibrate at a frequency that does not emulsify intraocular tissue. The device can further include an input configured to control the drive mechanism. The distal end effector can be configured to transition from an unexpanded state having a first outer diameter to an expanded state having a second outer diameter that is larger than the first outer diameter. The first outer diameter of the distal end effector in the unexpanded state can be sized to be surrounded by the outer sheath. The distal end effector can be self-expanding and transition toward the expanded state upon exposure from the outer sheath. The device can further include a core wire extending through a lumen of the treatment probe. The core wire can be configured to transition the distal end effector from the unexpanded state to the expanded state. The device can further include an irrigation channel for supplying irrigation fluid and a vacuum source for applying a vacuum through the device.

In an interrelated aspect, provided is a method of treating glaucoma including inserting a portion of an elongate shaft of an agitation device into the eye. The elongate shaft including a treatment probe including a distal end effector; and an outer sheath having a distal end region surrounding the distal end effector of the treatment probe. The method includes penetrating a trabecular meshwork with the distal end region of the outer sheath; exposing the distal end effector from the outer sheath; and vibrating the distal end effector within Schlemm’s canal to agitate intraocular tissue in contact with the distal end effector.

Vibrating the distal end effector to agitate intraocular tissue can loosen particulate from the trabecular meshwork. Vibrating the distal end effector includes actuating a drive mechanism operatively coupled to a proximal end of the treatment probe to cause oscillatory movement of the proximal end of the treatment probe. The drive mechanism can further include a piezoelectric crystal stack. The piezoelectric crystal stack can be driven at a frequency of 50 Hz - 40 kHz to vibrate the distal end effector. The distal end effector can vibrate at a frequency that does not emulsify intraocular tissue. Exposing the distal end effector from the outer sheath can include transitioning the distal end effector from an unexpanded state having a first outer diameter to an expanded state having a second outer diameter that is larger than the first outer diameter. The distal end effector can be self-expanding and transition toward the expanded state upon proximal withdrawal of the outer sheath from the distal end effector. The distal end effector can be actively expanded and transition toward the expanded state upon proximal withdrawal of the outer sheath from the distal end effector and proximal retraction of a core wire extending through a lumen of the treatment probe to axially compress the distal end effector. The method can further include supplying irrigation fluid through the elongate shaft; and applying vacuum through the elongate shaft.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1 is a diagram of the front portion of the eye;

FIG. 2 is a cross-sectional view of an implementation of a piezo-driven agitator device;

FIG. 3 is a side-view of an implementation of a piezo-driven agitator device;

FIG. 4 is a cross-sectional view of the device of FIG. 3;

FIG. 5 is a perspective view of the device of FIG. 3 with the outer housing shown as transparent;

FIG. 6 is a partially exploded view of the device of FIG. 3;

FIG. 7 is a perspective view of an implementation of a piezo-drive agitator device with the core wire retracted and the end effector sheathed;

FIG. 8 is a perspective view of the device of FIG. 7 with the core wire advanced and the end effector sheathed;

FIG. 9 is a perspective view of the device of FIG. 7 with the end effector unsheathed;

FIG. 10 is a detailed perspective view of the elongate shaft of a piezo-drive agitator device with the core wire retracted and the end effector sheathed;

FIG. 11 is a detailed perspective view of the elongate shaft of the device of FIG. 10 with the core wire advanced and the end effector sheathed;

FIG. 12 is a detailed perspective view of the elongate shaft of the device of FIG. 10 with an implementation of the end effector unsheathed;

FIG. 13 is a detailed perspective view of the elongate shaft of the device of FIG. 10 with another implementation of the end effector unsheathed;

FIG. 14A is a cross sectional view of the device of FIG. 7 with the core wire retracted and the end effector sheathed;

FIG. 14B is a detail view of the device in FIG. 14A taken at circle A;

FIG. 15A is a cross sectional view of the device of FIG. 7 with the core wire advanced and the end effector sheathed;

FIG. 15B is a detail view of the device in FIG. 15A taken at circle B;

FIG. 16A is a cross sectional view of the device of FIG. 7 with the end effector unsheathed;

FIG. 16B is a detail view of the device in FIG. 16A taken at circle C;

FIG. 17A is a cross sectional view of the device of FIG. 7 with the core wire retracted and the end effector sheathed;

FIG. 17B is a detail view of the elongate shaft in FIG. 17A taken at circle D;

FIG. 18A is a cross sectional view of the device of FIG. 7 with the core wire advanced and the end effector sheathed;

FIG. 18B is a detail view of the elongate shaft in FIG. 18A taken at circle E;

FIG. 19A is a cross sectional view of the device of FIG. 7 with an implementation of the end effector unsheathed;

FIG. 19B is a detail view of the elongate shaft of the device in FIG. 19A taken at circle F;

FIG. 20A is a cross sectional view of the device of FIG. 7 with another implementation of the end effector unsheathed;

FIG. 20B is a detailed view of the elongate shaft of the device in FIG. 20A taken at circle G;

FIG. 21 is a detailed cross sectional view of an elongate shaft of a device without a core wire;

FIG. 22A is a detailed cross sectional view of the elongate shaft of the device of FIG. 21 without a core wire and the end effector sheathed;

FIG. 22B is the elongate shaft of FIG. 22A following deployment of the end effector;

FIG. 23 is a detailed cross sectional view of an elongate shaft of a device with a core wire;

FIG. 24A is a detailed cross sectional view of the elongate shaft of the device of FIG. 23 with a core wire and the end effector sheathed;

FIG. 24B is the elongate shaft of FIG. 24A with the end effector unsheathed prior to deployment.

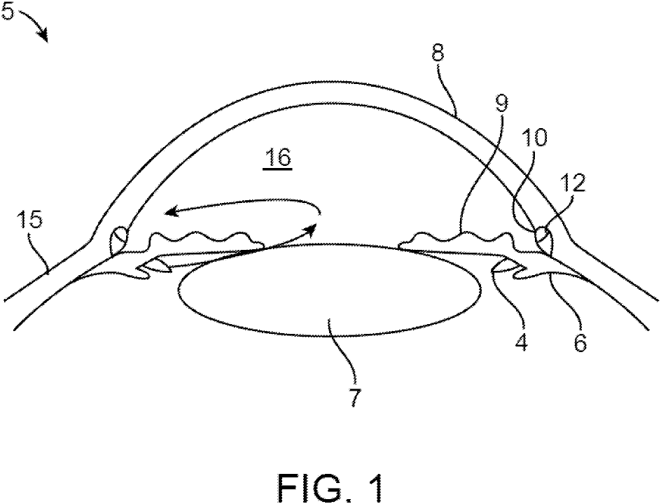

It should be appreciated that the drawings are for example only and are not meant to be to scale. It is to be understood that devices described herein may include features not necessarily depicted in each figure.

### DETAILED DESCRIPTION

Disclosed is a fully hand-held device for increasing aqueous humor outflow for the purpose of controlling intraocular pressure (IOP). More particularly and as will be described in detail below, the devices described herein involve mechanical vibration of a treatment probe positioned through the trabecular meshwork within Schlemm’s canal to enhance part of the natural drainage pathways of the eye by agitating intraocular tissues, loosening, breaking free, and/or removing built up particulate from the trabecular meshwork and within Schlemm’s canal. The treatment probe improves outflow through the trabecular meshwork so that aqueous can travel out of the anterior chamber and into Schlemm’s canal for improved control of IOP.

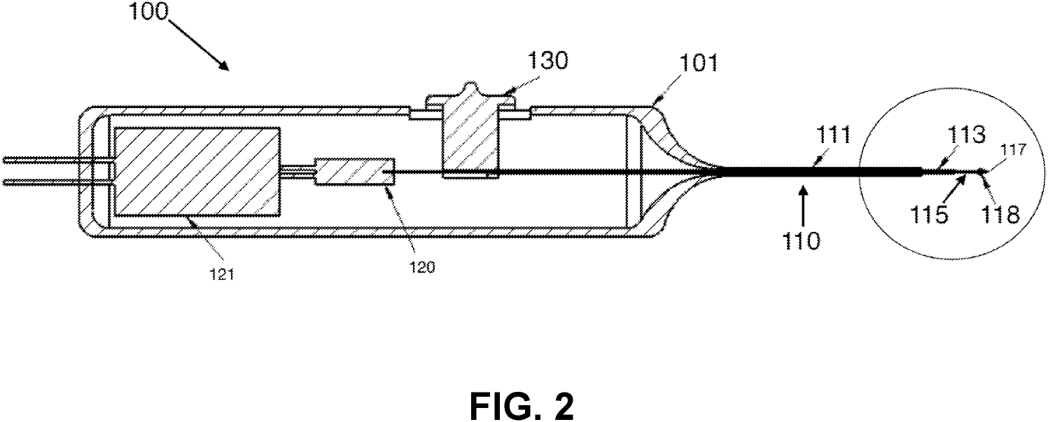

FIGS. 2, 3, and 7 are schematics illustrating implementations of agitator devices 100. The device 100 can include a handle or outer housing 101 having a proximal end region and a distal end region. The proximal end region is configured to be held by a user during use whereas the distal end region is configured to be inserted at least partially within the eye 5. An elongate shaft 110 projects distally from the distal region of the outer housing 101. At least the distal end of the elongate shaft 110 is sized to be inserted into the anterior chamber 16 of the eye 5 such as through a corneal incision. The elongate shaft 110 can include an outer-most introducer tube 111, an outer sheath 113 extending through and movable relative to a lumen 112 of the introducer tube 111, and an inner treatment probe 115 having a distal end effector 118 extending through and movable relative to a lumen 114 of the outer sheath 113. The outer-most introducer tube 111 can be integral with the outer housing 101. The elongate shaft 110 can also include a core wire 117 extending through and moveable relative to a lumen 116 of the inner treatment probe 115. The outer-most introducer tube 111 is configured to penetrate the eye 5, such as through a corneal or scleral penetration, and access the anterior chamber 16 of the eye 5. The outer sheath 113 and core wire 117 can be advanced relative to a distal end of the outer-most introducer tube 111 so as to be inserted into the trabecular meshwork 10. The core wire 117 can be further advanced relative to the outer sheath 113 deeper into the trabecular meshwork 10 and help guide outer sheath 113 deeper into the trabecular meshwork 10. The outer sheath 113 can then be retracted relative to the inner treatment probe 115 so as to unsheathe the distal end effector 118. Upon actuation of the device 100, the distal end effector 118 can agitate the surrounding intraocular tissue such as the trabecular meshwork 10 and Schlemm’s canal 12 so as to loosen and remove particulate from the trabecular meshwork 10 and Schlemm’s canal 12. The inner treatment probe 115 is operatively coupled to a piezo drive mechanism 120 configured to vibrate the end effector 118 of the probe 115. The drive mechanism 120 can include a piezoelectric stack 122, configured to oscillate and transmit vibrations to the end effector 118, and can be used with a control board 121, which will be described in more detail below. The device 100 can have one or more inputs that can be used to control the various functions of the device 100, which will be described in more detail below.

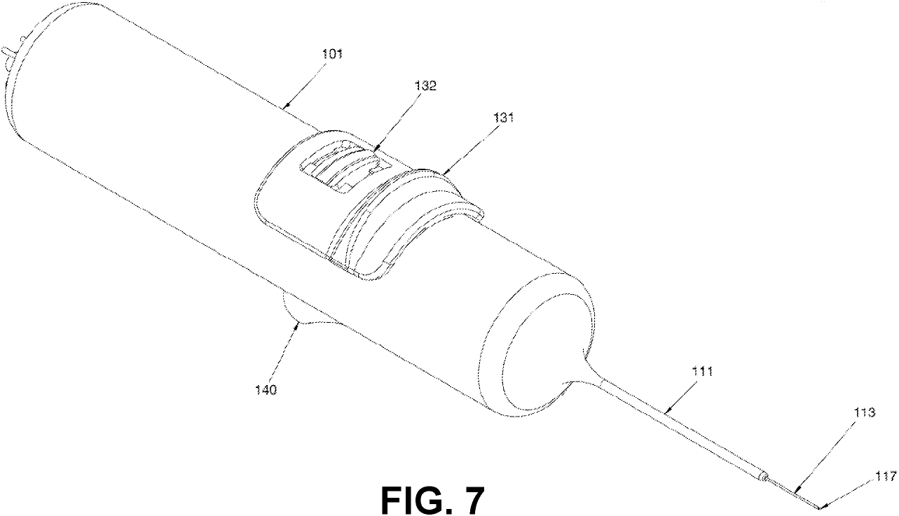

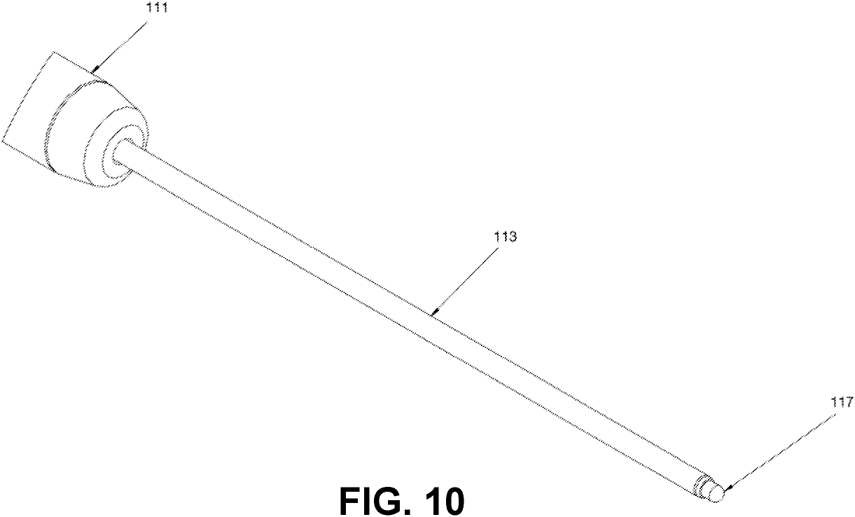

As mentioned above, the elongate shaft 110 can include the introducer tube 111, best seen in FIGS. 7-9. The introducer tube 111 can be permanently attached to the outer housing 101, but can be detachable in some embodiments. In a method of using the device 100, an incision or puncture can be made in the eye 5 and the introducer tube 111 inserted to access the interior of the eye 5. Introducer tube 111 can be rigid so as to not deform during insertion into the eye 5 and holds open an access point through which other components of the elongate shaft 110 can be inserted, such as the outer sheath 113, the inner treatment probe 115, and the core wire 117. Introducer tube 111 can be made out of a material with suitable stiffness/rigidity for this purpose, including metals such as stainless steel or Nitinol, or plastic. The length of the introducer tube 111 can vary, but is generally between about 2 mm and about 10 mm. The introducer tube 111 can have an outer diameter suitable for insertion in the eye (e.g., about 0.5 mm - 1.5 mm). The inner diameter of the introducer tube 111 is sufficient to receive the outer sheath 113, for example, between about 0.1 mm and about 0.14 mm. The outer sheath 113 is best seen in FIGS. 10-13. The outer sheath 113 can be positioned within the lumen 112 of the introducer tube 111. The outer sheath 113 can have an outer diameter that is about 0.2 mm - 0.4 mm and an inner diameter sufficient to receive the inner treatment probe 115 and end effector 118 therein, for example, between about 0.1 mm - 0.2 mm. The length of the outer sheath 113 can vary, but is generally between about 25 mm - 75 mm. The outer sheath 113 can extend a distance beyond a distal end of the introducer tube 111 such as at least by about 5 mm up to about 20 mm. The outer sheath 113 can be movable relative to the introducer tube 111 such as by being retracted relative to the introducer 111.

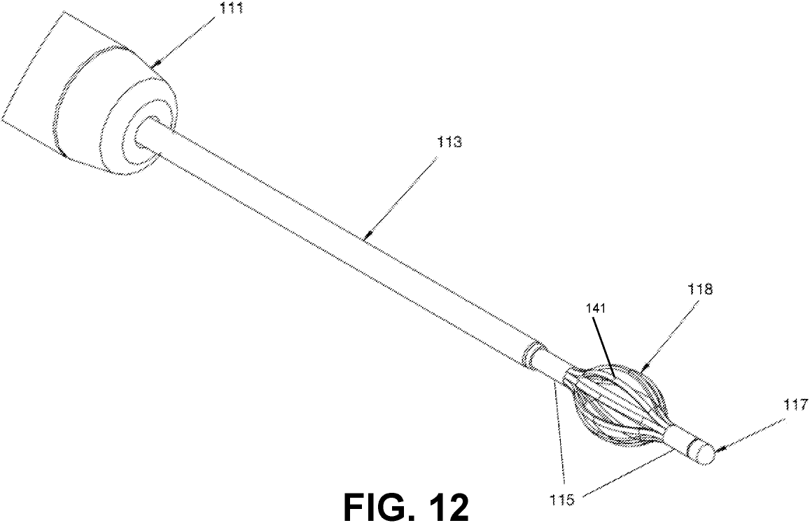

The inner treatment probe 115 is best seen in FIGS. 12, 13, 19, and 20. The inner treatment probe 115 is positioned within the lumen 114 of the outer sheath 113. The inner treatment probe 115 can have an outer diameter of about 0.10 mm - 0.20 mm. The treatment probe 115 can have an inner diameter that is about 0.05 mm - 0.15 mm. The length of the inner treatment probe 115 can vary, for example, between at least about 50 mm up to about 150 mm. The inner treatment probe 115 can extend relative to the outer sheath by at least about 1 mm up to about 20 mm. The inner treatment probe 115 can be moved relative to the outer sheath 113 to achieve the relative extension beyond the distal end of the outer sheath 113, for example, by moving the probe 115 in a distal direction. The inner treatment probe 115 can be fixed relative to the outer sheath 113 and the relative extension of the probe 115 beyond the distal end of the outer sheath 113 achieved by retracting the outer sheath 111 proximally relative to the probe 115. In still further implementations, both the probe 115 and the sheath 111 can be movable so that relative extension between them can be achieved by distal extension and/or proximal retraction.

In operation of the device 100, the outer sheath 113 can be advanced past the distal end of the introducer tube 111 within the eye 5, into at least the trabecular meshwork 10. The outer sheath 113 houses the inner treatment probe 115 and core wire 117, and keeps the end effector 118 sheathed. FIGS. 10-13 illustrate the outer sheath 113 advanced past the distal end of the introducer tube 111 and housing the inner treatment probe 115 and core wire 117. Outer sheath 113 can be less rigid than the introducer tube 111, and can be made of a semi-flexible material such as stainless steel, Nitinol, or plastic, among other materials. The outer sheath 113 can be straight, as depicted in the figures, or can be a curved shape to match the curvature of the eye. The distal tip of the outer sheath 113 can be tapered to allow easier insertion into the eye.

The end effector 118 is located on a distal end region of the inner treatment probe 115, for example, a distance proximal to the distal-most tip of the inner treatment probe 115. The end effector 118 location along the length of the inner treatment probe 115 can vary. In some implementations, the end effector 118 is located at least about 0.1 mm from the distal-most tip of the probe 115. The end effector 118 can be located up to about 5 mm from the distal-most tip of the probe 115. The end effector 118 can be about 0.5 - 2.0 mm along a length of the probe 115. As will be described in more detail below, the end effector 118 can be an expandable element configured to increase from an outer diameter of about 0.1 mm - 0.2 mm to an expanded outer diameter of about 0.5 mm - 2 mm.

In operation of the device 100, the inner treatment probe 115 can be advanced up to about 3 mm past the distal end of the outer sheath 113 and inserted through the trabecular meshwork 10 into Schlemm’s canal 12. The inner treatment probe 115 can penetrate and/or cannulate the canal 12. Advancement of inner treatment probe 115 past the distal-most end of outer sheath 113 unsheathes the end effector 118 on the distal end region of the inner treatment probe 115. In embodiments with a core wire 117 present, the inner treatment probe 115 can be hollow and include a distal surface at the distal tip of the inner treatment probe 115, which a proximal surface of the distal tip of the core wire 117 abuts against. The interaction between these surfaces can be necessary to effect expansion of the end effector 118, which is described in more detail below. The inner treatment probe 115 can be flexible in order to achieve cannulation of Schlemm’s canal 12, and can be made of Nitinol or plastic.

Agitation of tissues in and around Schlemm’s canal 12 by the inner treatment probe 115 and the end effector 118 can loosen and free particulate in the surrounding intraocular tissues. The inner treatment probe 115 can extend a distance beyond the inner wall of Schlemm’s canal (i.e., the trabecular meshwork) to apply vibrations of the end effector 118 to agitate the surrounding tissues. The trabecular meshwork can vary in thickness from about 50 um to about 150 um. The inner treatment probe 115 can extend through the trabecular meshwork (at least about 150 um) and a distance into Schlemm’s canal to apply vibration of the end effector 118 to the site. In some implementations, the inner treatment probe 115 is flexible and configured to curve so as to travel along a length of Schlemm’s canal 12 so that the vibration applied by the end effector 118 is a distance away from the site of penetration of the end effector 118 through the trabecular meshwork. The inner treatment probe 115 can penetrate the trabecular meshwork at least once during a treatment, preferably more than once at different locations of Schlemm’s canal. The inner treatment probe 115 can penetrate the trabecular meshwork at multiple points around the circumference of the eye including at least 2 penetrations 180 degrees apart, at least 3 penetrations 120 degrees apart, at least 4 penetrations 90 degrees apart, and so on so that agitation of the end effector 118 is performed multiple times during a procedure. The multiple penetrations of the probe 115 (and end effector 118) through the trabecular meshwork can also be grouped at a particular location around the circumference of the eye, including one, two, three, four, or more penetrations of the trabecular meshwork at a single treatment location along the circumference of the eye. The multiple penetrations and thus, multiple agitations can be performed at more than a single treatment location spaced around the circumference of the eye as described above (i.e., a first group at a first location, a second group at a second location, a third group at a third location, and so on). Depending on the outflow achieved, multiple insertions of the probe 115 and vibrations of the effector 118 into Schlemm’s canal can be performed to treat the entirety of Schlemm’s canal.

Loosening particulate from the surrounding intraocular tissues, such as the trabecular meshwork 10 and Schlemm’s canal 12, is achieved through gentle agitation caused by vibration of the end effector 118 against tissues with minimal tissue damage. Preferably, the vibration of the end effector 118 is gentle enough to avoid destroying tissue it contacts, but great enough to be transmitted into the surrounding tissue to cause gentle agitation of the tissue without destroying or emulsifying the tissue. The end effector 118 is configured to vibrate against the walls of Schlemm’s canal 12 and/or the trabecular meshwork, thereby allowing the vibrational motion to propagate into the surrounding tissue. Vibrational motion of the end effector 118 can be propagated into the surrounding tissue at an effective depth of 0.01 mm to 0.4 mm, and the propagated vibrational motion can be concentrated near the end effector 118. The vibration of the end effector 118 can be transmitted to tissues by direct contact with walls of Schlemm’s canal including the outside wall of the canal and/or the inner wall of the canal (i.e., the trabecular meshwork). Vibrations of the tissues are transmitted into agitation of the trapped particulate, subjecting the particulate to stress and shear, which helps break up the particulate into small pieces that can be freed from the tissue. Loosening and freeing the particulate from these tissues enhances the action of the naturally present drainage pathways in the eye. The end effector 118 vibration is dependent on the frequency of the piezoelectric stack 122. Increasing the frequency of the drive 120 can increase the force or energy of the vibration at the end effector 118 that is transmitted to agitate the tissues and particulate. In some implementations, a user can apply maximum energy to destroy a tissue such as by emulsification. For example, a user can destroy one or more regions of the trabecular meshwork using the end effector 118. Frequency modulation and/or geometry of the end effector 118 can also be selected to prevent the device from destroying tissues. The drive frequency of the piezo drive mechanism 120 is discussed in more detail below.

End effector 118 is best seen in FIGS. 12, 13, 19A-19B, and 20A-20B. The end effector 118 can be located proximal to the distal-most end of the inner treatment probe 115 and is configured to vibrate when the piezo drive mechanism 120 is activated. The piezo drive mechanism 120 can be coupled to the proximal end of probe 115. Oscillation of the piezoelectric crystal stack 122 are transmitted from the proximal end of the treatment probe 115 distally to cause vibration of the end effector 118. Vibration of the end effector 118 can cause contact against surrounding tissues at the treatment site thereby agitating those particulates as discussed elsewhere herein.

The end effector 118 can be an uncut tube, round, or non-round rod or wire. In some implementations, the end effector 118 forms a surface geometry near the distal end region of the treatment probe 115 that is generally discontinuous. For example, the end effector 118 can incorporate a plurality of flexible tines 141 around the circumference of the shaft. The tines 141 of the end effector 118 can be thin and flexible, and are configured to enhance agitation of particulate. In some implementations, the end effector 118 is located just proximal to the distal-most end of the probe 115. The end effector 118 can be formed by two cuts on opposing sides of the hollow shaft of the treatment probe 115 creating two tines 141, or three cuts forming three tines 141, and so on so that the end effector 118 is formed by one, two, three, four, five or more pairs of tines 141. Each of the tines 141 can have a width such that a space is formed between them upon expansion of the end effector 118. The width of each of the tines 141 can be between about 0.05 mm and about 0.15 mm depending on the number of tines 141 of the end effector 118. Each of the tines 141 can have a geometry that is generally rounded as a round wire. Alternatively, each of the tines 141 can be flattened on an outer and/or inner surface as a flattened ribbon. The edges between the flattened surfaces can be square or rounded so as to avoid being sharp. The wall thickness of each of the tines 141 can be uniform or non-uniform along a length of the end effector 118.

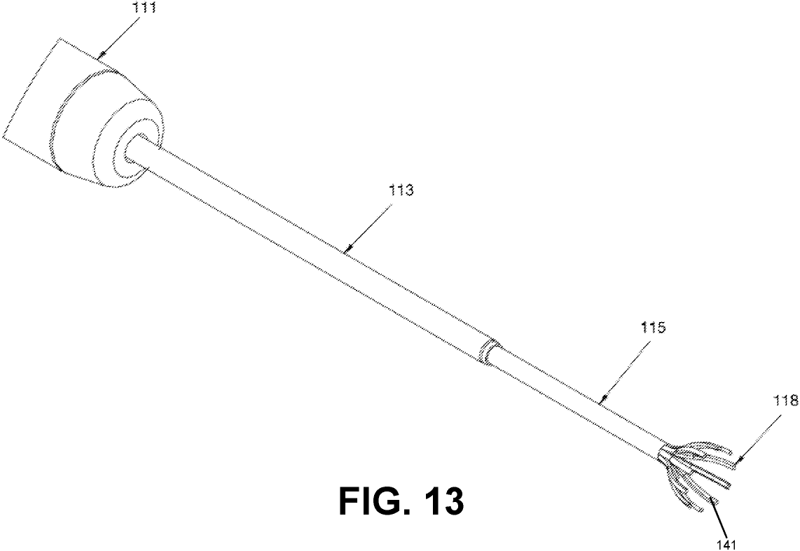

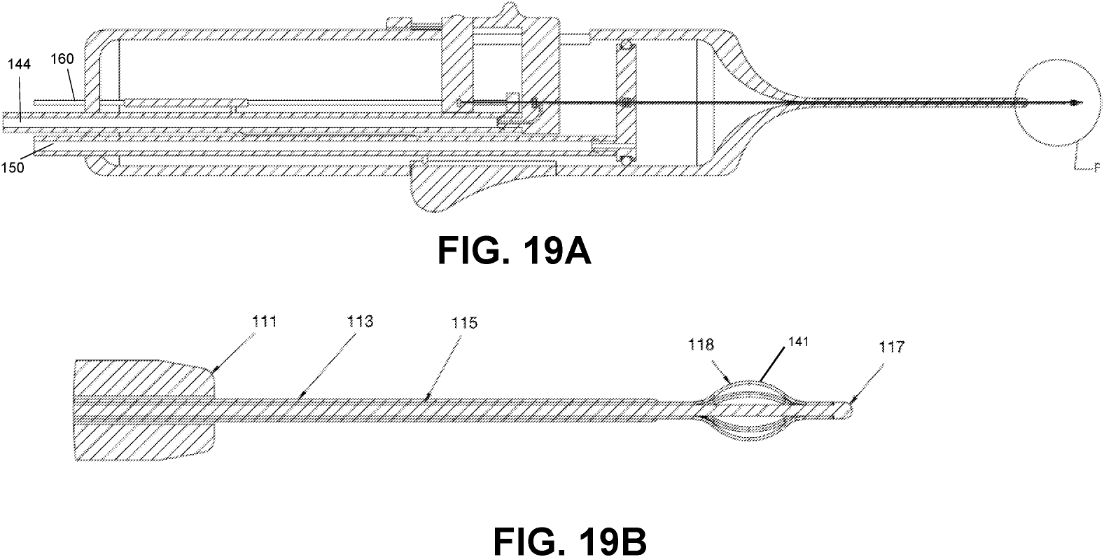

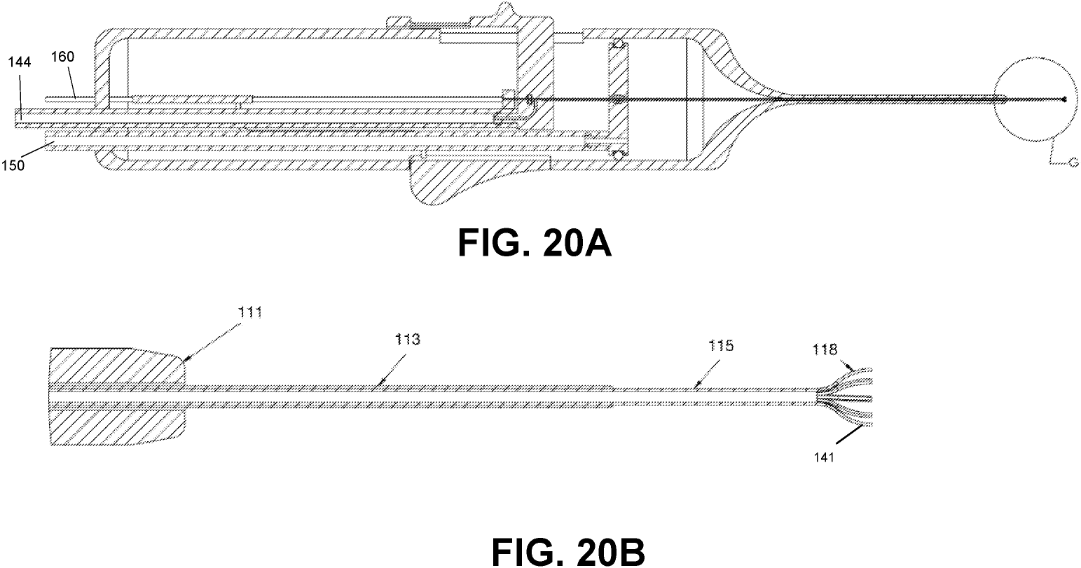

The shape of the tines 141 can provide a specific overall configuration to the expanded end effector 118. The end effector 118 can have a variety of shapes, including a fully tubular cage-like or whisk-like expanded shape seen in FIGS. 12 and 19A-19B or an open-ended expanded shape seen in FIGS. 13 and 20A-20B. Other shapes can also be used, such as a straight rod, a balloon, or a cup. Certain shapes can increase vibrational movement, enhancing agitation of intraocular tissues it contacts and any particulate. The tines 141 can create the spheroid-like geometry such as shown in FIGS. 12, 19A-19B due to the end effector 118 being located a distance proximal to the distal-most tip of the shaft. The distal ends of the tines 141 can approach the outer diameter of the probe 115 just distal to the end effector 118 and the proximal ends of the tines 141 can approach the outer diameter of the probe just proximal to the end effector 118. The end effector 118 can form the distal-most tip of the inner treatment probe 115 as shown FIGS. 13, 20A-20B so that the end effector is open-ended forming an umbrella shape rather than a spheroid shape.

The end effector 118 can have an outer diameter of about 0.1-0.2 mm in the unexpanded configuration and an outer diameter of about 0.5 - 2.0 mm at its greatest dimension in the expanded configuration. Smaller diameters can be preferred for increased flexibility and vibrational movement, enhancing agitation of the intraocular tissues and particulate.

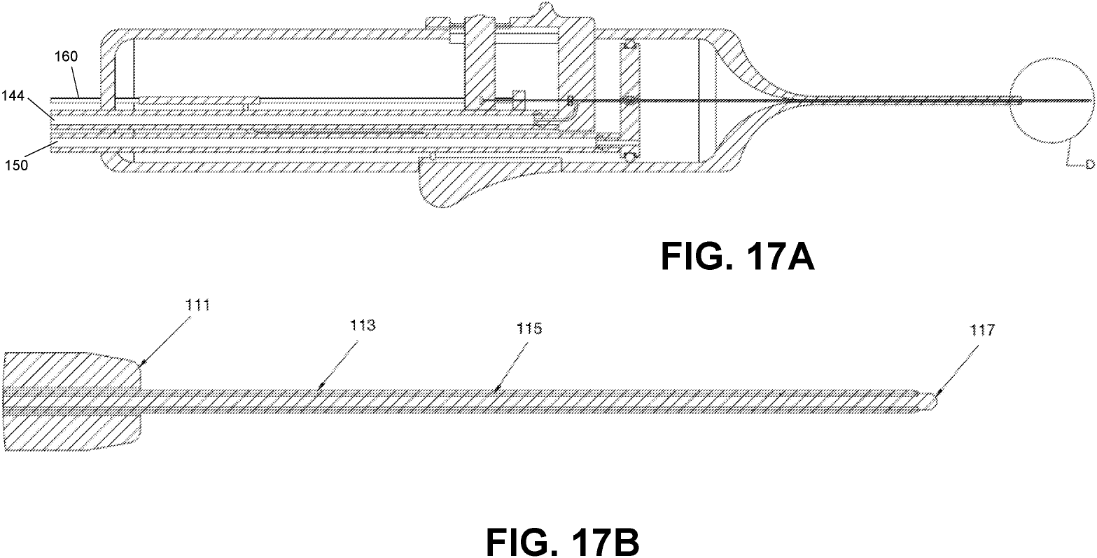

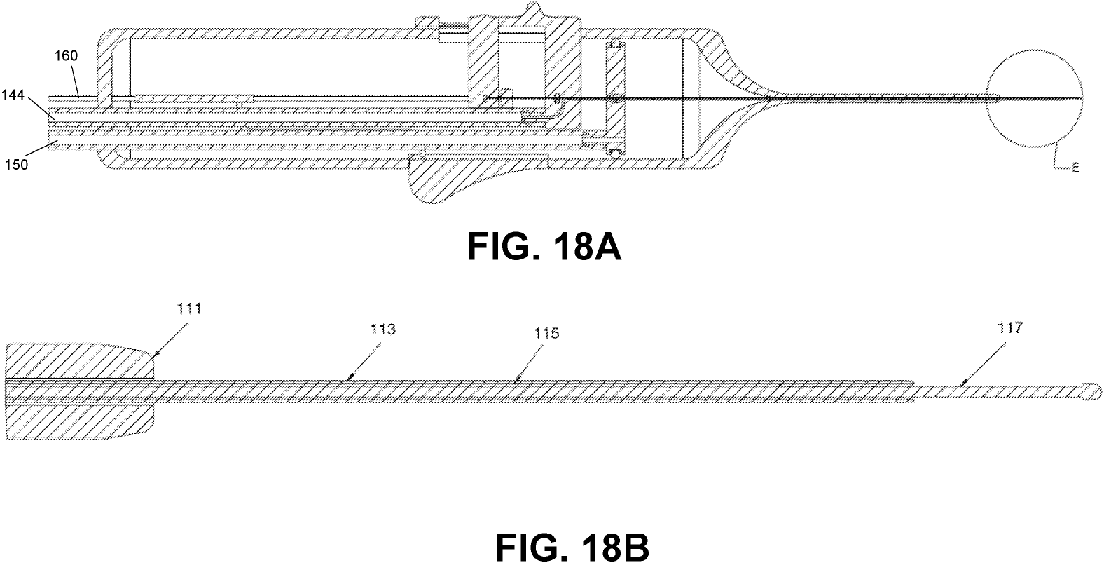

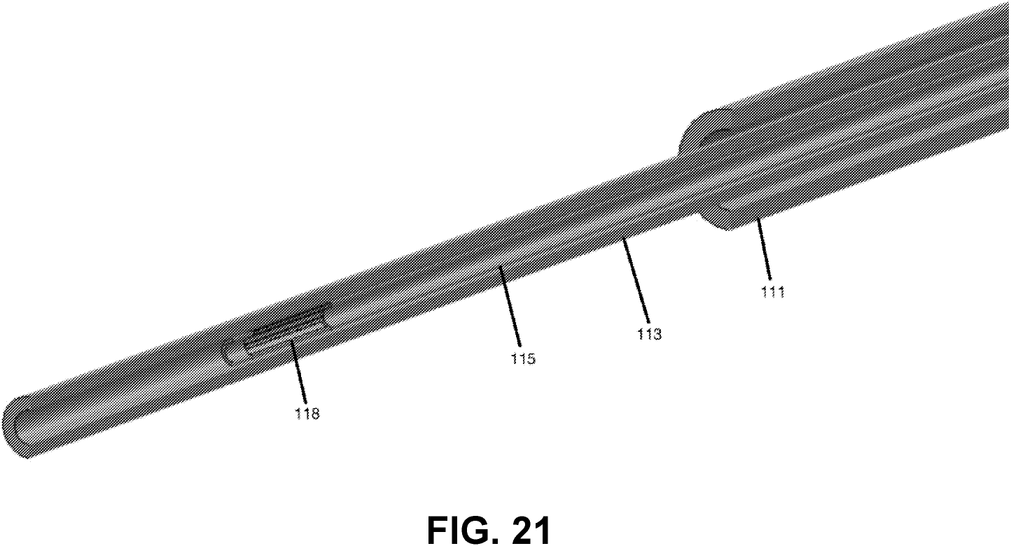

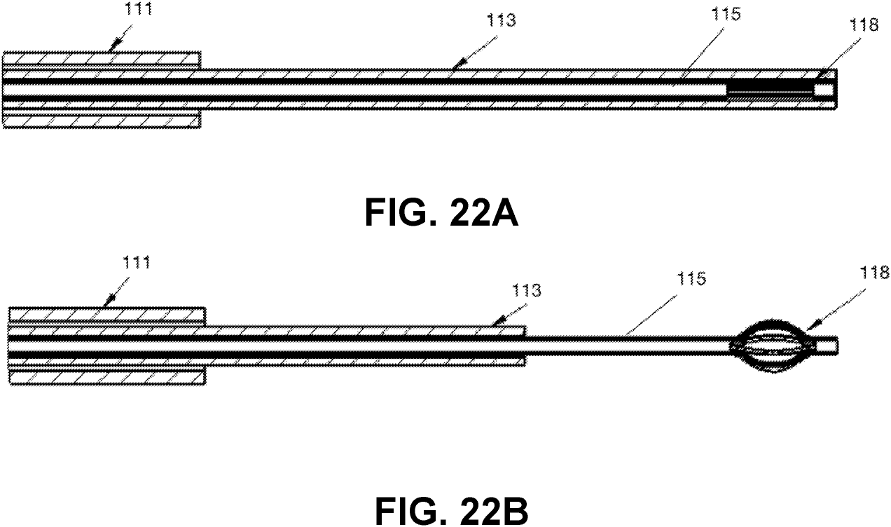

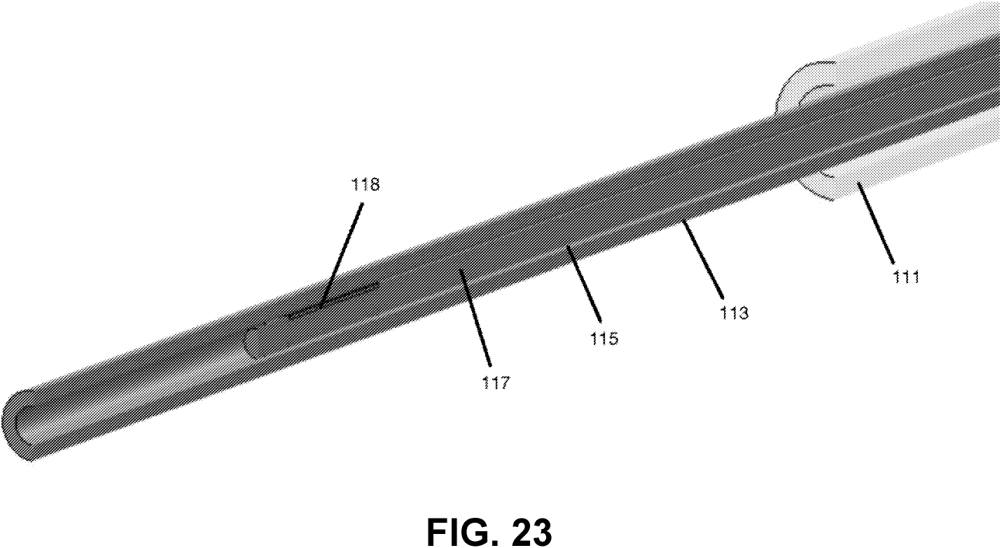

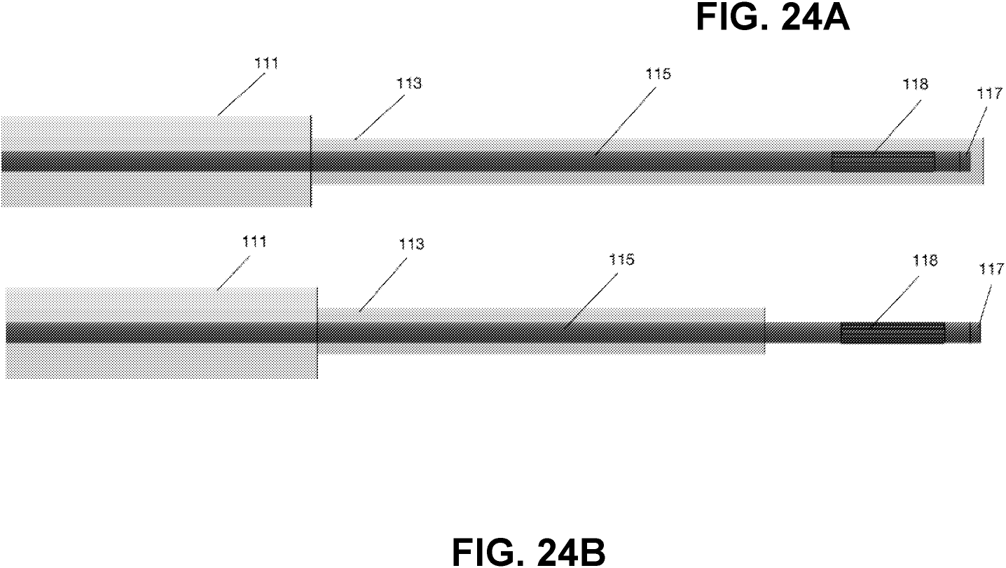

The end effector 118 can be self-expanding element configured to transition from the lower profile, unexpanded configuration to the higher profile, expanded configuration during treatment. Following treatment, the end effector can then be retracted into its unexpanded configuration from removal from the eye. The end effector 118 can be contained in the unexpanded configuration when positioned within and covered by the outer sheath 113, seen in FIGS. 10, 11, 17A-17B, 18A-18B, 21, and 23. Upon retraction of the outer sheath 113 (and/or advancement of the inner treatment probe 115) the end effector 118 near the distal end of the inner treatment probe 115 is uncovered and exposed allowing it to transition towards the expanded configuration, seen in FIGS. 12, 13, 19A-19B, and 20A-20B. As seen in FIGS. 13, 20A-10B, 21, and 22A-22B, the end effector 118 can be self-expanding, i.e. can automatically assume its expanded configuration by virtue of being uncovered from the outer sheath 113. This can be achieved by the end effector 118 being shape-set in the expanded configuration, and held in the unexpanded configuration by compression from the inner surface of outer sheath 113. FIGS. 22A-22B illustrate the transition from the unexpanded configuration to the expanded configuration in a self-expanding end effector 118 by retracting the outer sheath 113 and uncovering the inner treatment probe 115 and end effector 118. Alternatively, the end effector 118 can be actively expanded, as seen in FIGS. 12, 19A-19B, 23, and 24A-24B. In this embodiment, the inner treatment probe 115 can be hollow and include a distal surface at the distal tip of the inner treatment probe 115, which a proximal surface of the distal tip of the core wire 117 abuts against. Active expansion of the end effector 118 can be achieved by proximally retracting the core wire 117 to axially compress end effector 118, causing it to assume the expanded configuration. The end effector 118 pictured in FIGS. 12 and 19A-19B can employ a core wire 117. The end effector 118 pictured in FIGS. 13 and 20A-20B need not include a core wire 117 and can be shape-set to transition into the expanded configuration, but alternatively can be used with a core wire 117 that is not used to transition the end effector 118 into the expanded configuration. The expanded configuration increases the vibrational movement of the end effector 118 and enhances agitation of particulate. The self-expanding end effector 118 can be made of a shape-set material such as Nitinol, and the actively-expanding end effector 118 can be made from stainless steel or plastic.

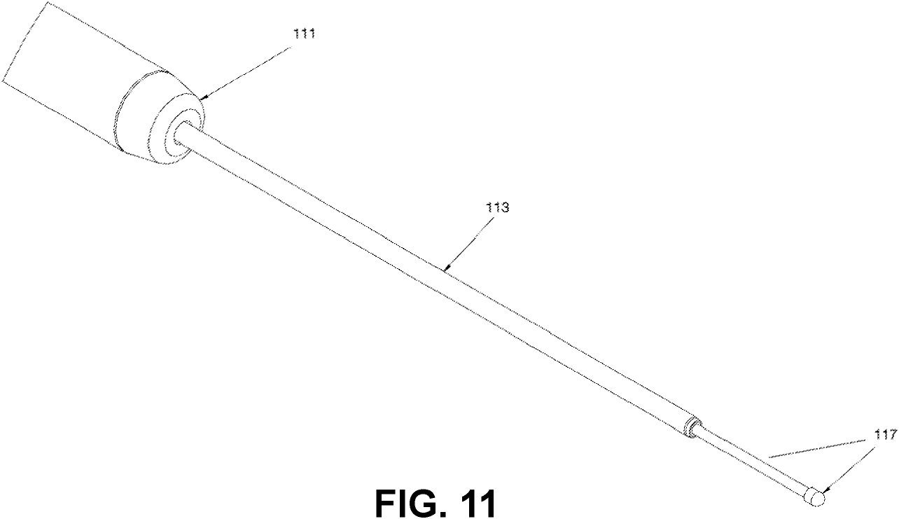

The core wire 117 is best seen in FIGS. 10-12, and 17A-19B. The core wire 117 can be located within the lumen 116 of inner treatment probe 115 and can be optionally included or not included. A core wire 117 can be present to activate expansion of the active-expansion end effector 118. A core wire 117 is not necessary to activate expansion of the self-expanding end effector 118, but can be present, and may help keep the shape-set material of the end effector 118 from collapsing inward. FIG. 10 shows the distal tip of the core wire 117 at the distal tip of the elongate shaft 110. The distal tip of core wire 117 can protrude slightly, be flush, or be subflush to the distal end of outer sheath 113. The core wire 117 can be inserted through the trabecular meshwork 10 along with the outer sheath 113. The core wire 117 can then be advanced relative to the outer sheath 113 and penetrate deeper through the trabecular meshwork 10 to help guide the outer sheath 113 deeper into Schlemm’s Canal. FIG. 11 shows the core wire 117 advanced distally, exposing a thin inner core and the larger distal tip of the core wire 117. The distal tip of core wire 117 can have a rounded or domed shape to prevent tissue damage. FIG. 12 shows the distal tip of the core wire 117 abutting against the distal tip of the inner treatment probe 115 and end effector 118. The proximal surface of the distal tip of core wire 117 can abut against a distal surface of the distal tip of inner treatment probe 115. This interaction between the surfaces allows retraction of the core wire 117 to axially compress the inner treatment probe 115 and end effector 118 in embodiments where the end effector 118 is actively expanded. The core wire 117 can increase stiffness of the inner treatment probe 115, and can be a solid or hollow rod, and can have a diameter from 0.002 inches to 0.006 inches. The core wire 117 can be made from a semi-flexible material such as stainless steel, Nitinol, or plastic, among other materials.

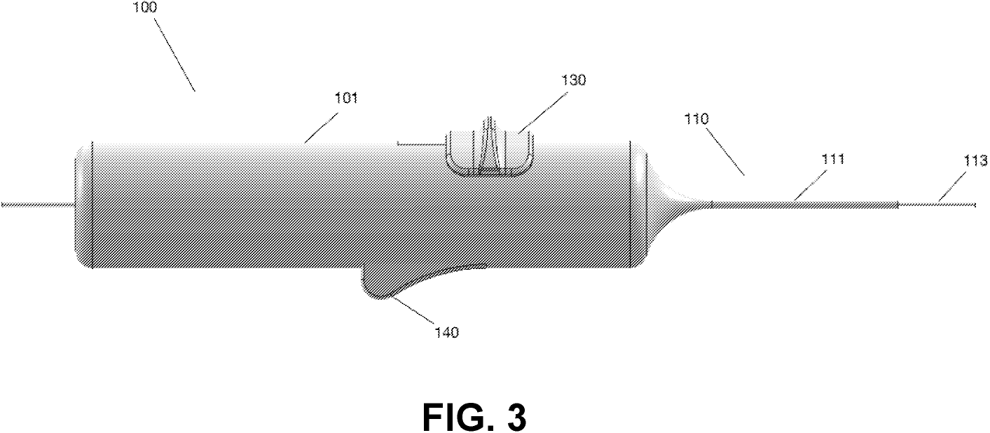

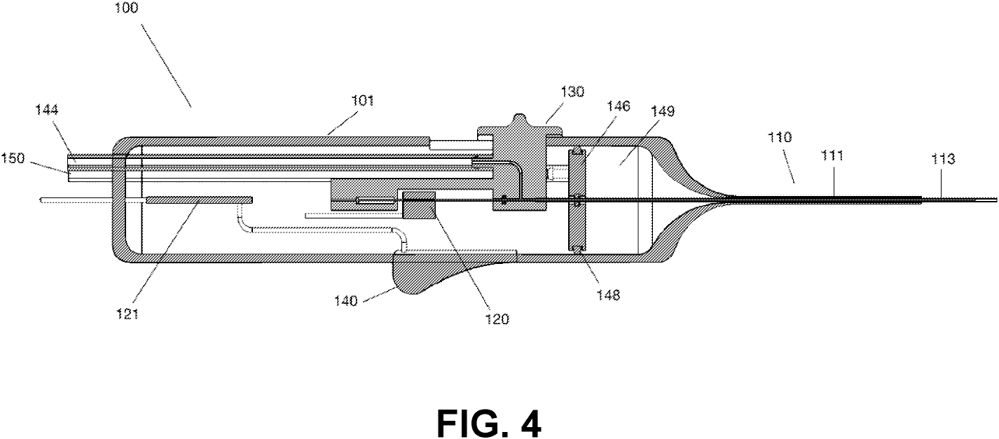

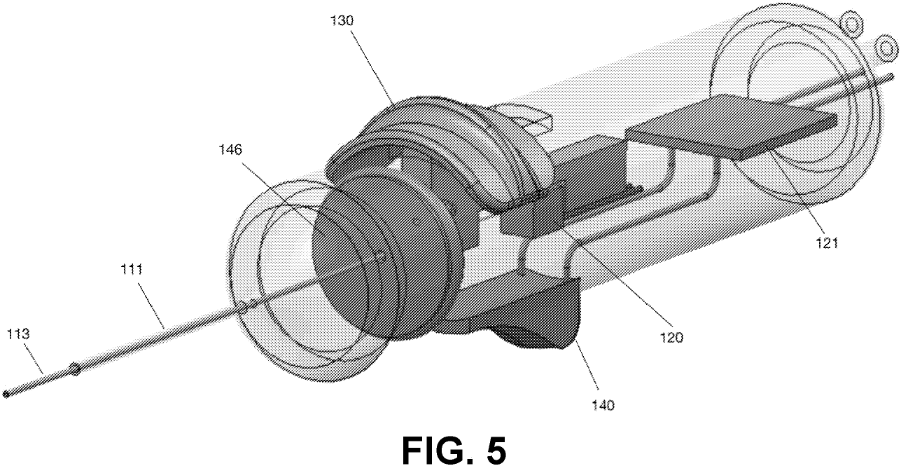

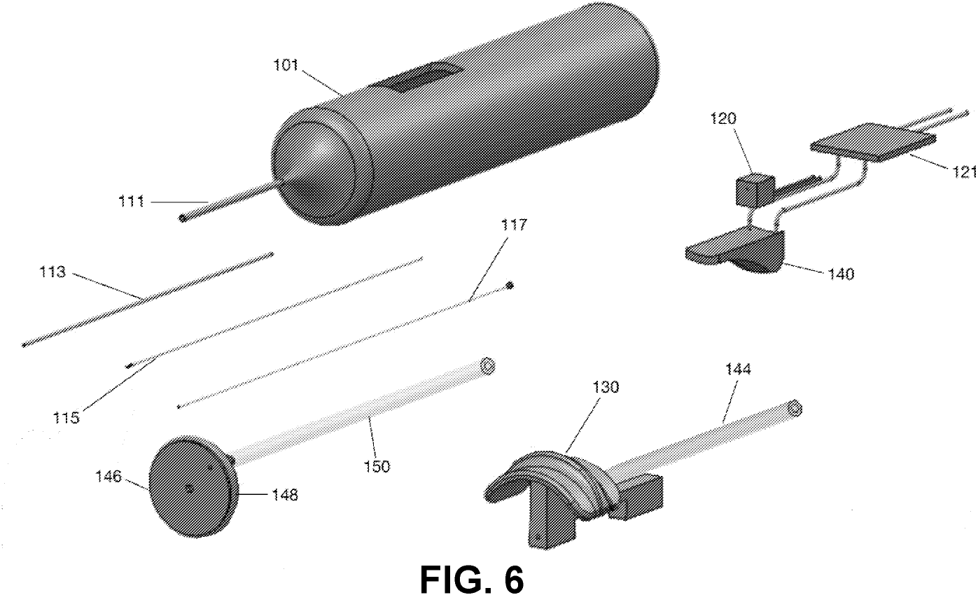

The device 100 can have one or more inputs that can be used to control the various functions of the device 100. The inputs can be sliders, triggers, buttons, foot pedals, touch screens, or any other suitable input. FIG. 2 shows a device 100 with a slider 130 to control advancement and retraction of the various components of the elongate shaft 110 and vibration of the end effector 118. Although the device 100 in FIG. 2 is shown with a single slider 130, the device 100 can have multiple inputs that control different functions, as shown in FIG. 3 and also FIG. 7. As shown in FIGS. 3 and 4, the outer housing 101 can include a slider 130, which the user can use to advance and retract the various components of the elongate shaft 110. As shown in FIGS. 3, 4, and 7, the outer housing 101 can include a trigger 140, which the user can use to actuate the device 100 and cause vibration of the end effector 118. Additional inputs can be included on the housing 101 and/or otherwise connected to device 100 in order to actuate additional features such as vacuum, irrigation, and more. For example, the device 100 can incorporate a foot switch to control vacuum and/or irrigation or an additional input(s) on the housing 101 of the device 100 can be used to control vacuum and/or irrigation. The input configured to activate one of the functions of the device can be configured to additionally activate another function of the device depending on degree of actuation. Activation of the piezo can be linked to activation of vacuum so as to only apply vacuum during piezo cycles.

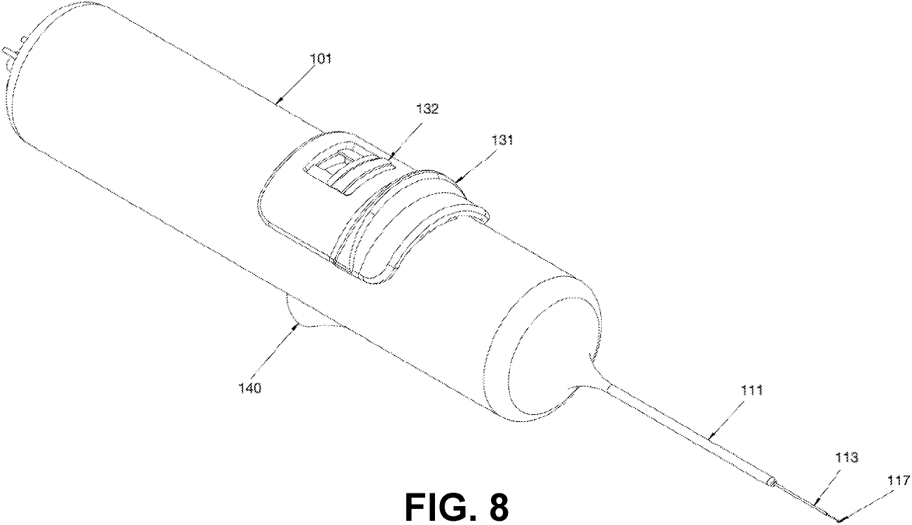

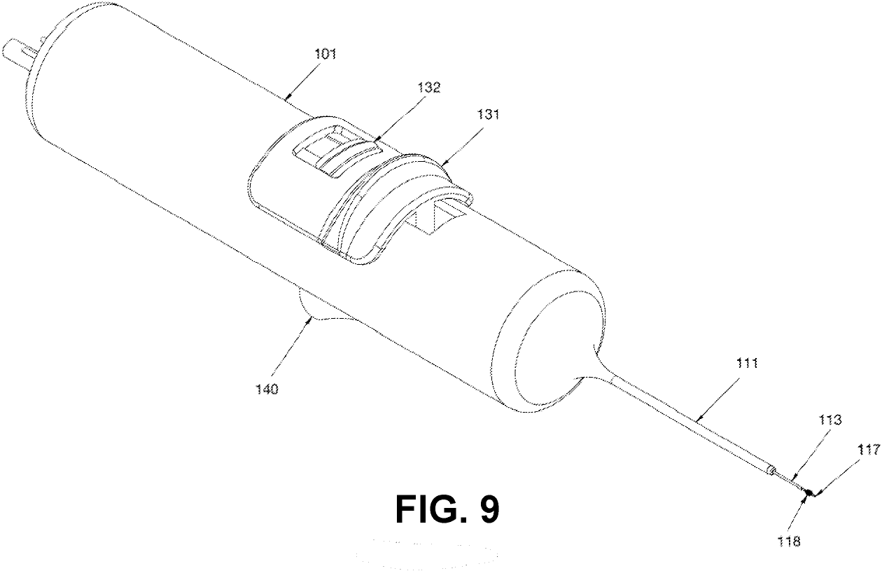

As seen in FIGS. 7-9, slider 130 can be a primary slider 131 and a secondary slider 132. Primary slider 131 can be attached to the outer sheath 113 and can slide axially along the outer housing 101, allowing advancement and retraction of outer sheath 113. Secondary slider 132 can be attached to the core wire 117, allowing core wire 117 to move independently of outer sheath 113 and be advanced distally or be retracted. FIG. 7 depicts device 100 with the end effector 118 sheathed and the core wire 117 in its default retracted state. Distally advancing the second slider 132 distally advances the core wire 117 past the distal end of the outer sheath 113, shown in FIG. 8. As depicted in FIG. 9, proximal retraction of primary slider 131 retracts both the outer sheath 113 and the core wire 117, allowing the inner treatment probe 115 and the end effector 118 to be unsheathed. Once the end effector 118 is unsheathed and in the expanded configuration, trigger 140 can be depressed to activate piezo drive mechanism 120. The degree of trigger 140 depression can control the frequency of vibration, the amplitude of vibration, or both.

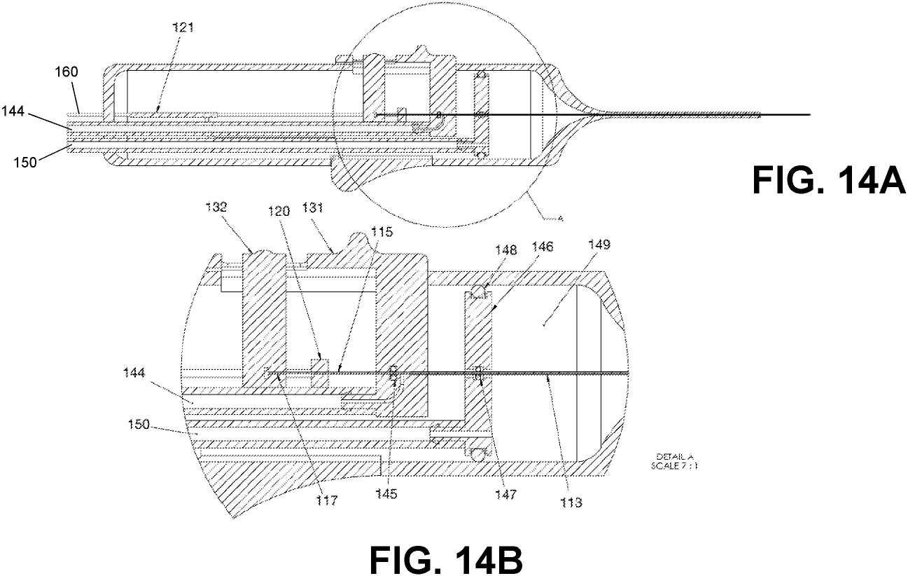

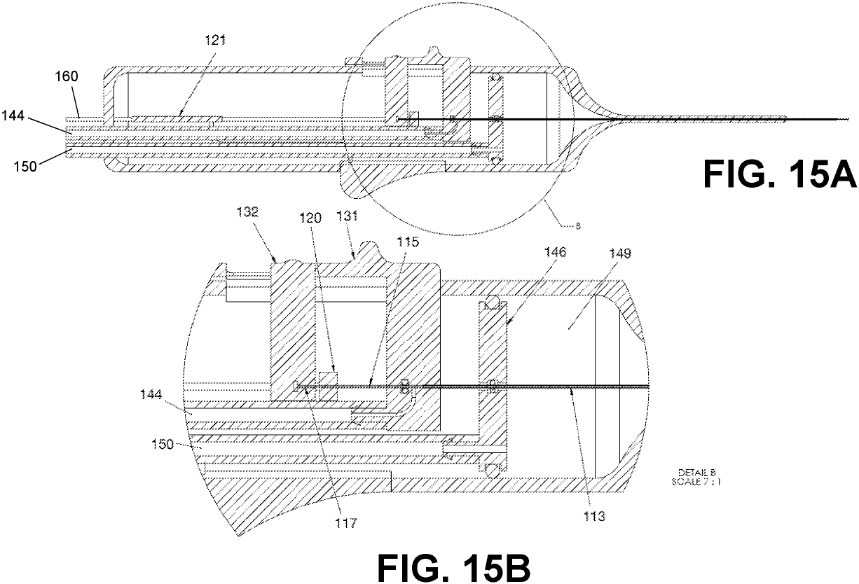

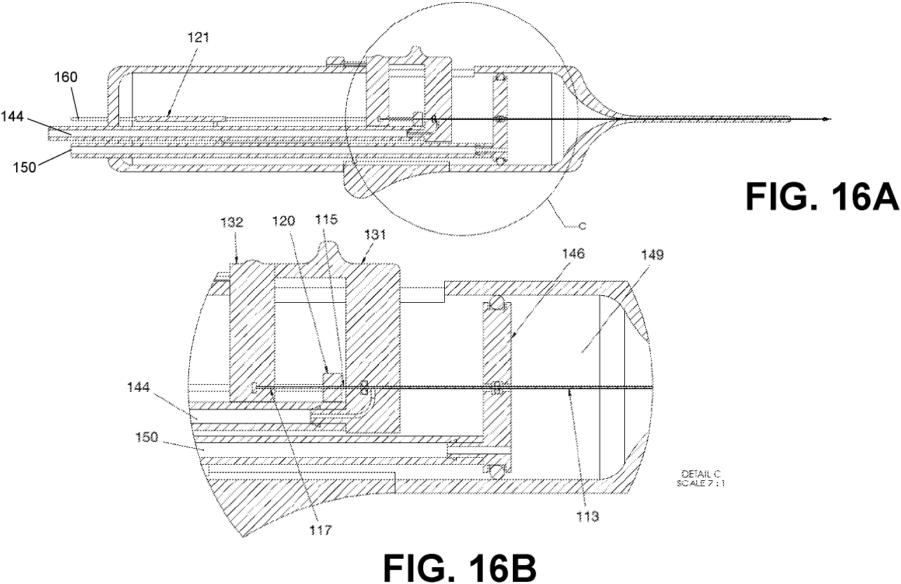

As seen in FIGS. 14A-14B, 15A-15B, and 16A-16B, the proximal end of the core wire 117 can be grounded axially within the secondary slider 132, such that the two components are axially mated. The proximal end of outer sheath 113 can be axially fixed to the primary slider 131 such that as the primary slider 131 moves proximally or distally, the outer sheath 113 accordingly moves proximally or distally. The primary slider 131 can also house an inner probe seal 145, which fluidically isolates the annular space between the inner treatment probe 115 and the outer sheath 113 such that either vacuum or irrigation can be applied to the space. The application of vacuum and/or irrigation will be described in further detail below.

The drive mechanism 120 can include a piezoelectric crystal stack 122 that can expand and contract based on applied voltage creating oscillation that is transmitted along the inner treatment probe 115 to cause vibration of the end effector 118. The piezoelectric drive mechanism 120 is configured to amplify the motion of the piezoelectric material of the stack 122 into sufficient vibration of the end effector 118. The resulting movement of the end effector 118 may be described herein as vibration, agitation, oscillation, or any other sort of motion that results from the proximal end of the inner treatment probe 115 being driven by the piezoelectric crystal stack 122.

As shown in FIGS. 14A-14B, 15A-15B, and 16A-16B, the piezoelectric crystal stack 122 can be physically coupled to the proximal end of inner treatment probe 115 such that the oscillations travel along the length of the inner treatment probe 115, resulting in vibration of the end effector 118 at the distal end region of the probe 115. The piezoelectric crystal stack 122 can be ring-shaped and have an inner diameter sized to receive the outer diameter of the proximal end of the inner treatment probe 115. The proximal end of the treatment probe 115 can be rigidly connected to the crystal stack 122 and move with the stack 122. The expansion and contraction of the crystal stack 122 can generate vibrations of the end effector 118 at varying frequencies. The piezoelectric stack 122 can change with varying voltage including alternating current or DC variable voltage. In an implementation, an alternating current (e.g., 50 Hz to 40 kHz) applied to the piezoelectric stack 122 causes the stack 122 to expand and contract. As the stack 112 expands and contracts, the proximal end of the inner treatment probe 115 moves causing the distal end effector 118 to move as well. Movement of the end effector 118 at the distal end region of the probe 115 can be greater than movement at the proximal end of the probe 115. The inner treatment probe 115 can be driven directly (with or without amplifying components) by the piezoelectric crystal stack 122 in a non-resonant manner at a frequency that is between about 50 Hz up to about 40 kHz. There can, but need not be a 1:1 relationship between the piezoelectric stack 122 and end effector frequencies. Where the frequency of the crystal stack 122 is described as being 50 Hz, the end effector frequency can also be 50 Hz. Alternatively, there may be certain resonant frequencies that multiply the frequency of the end effector 118 to be greater than the frequency of the stack 122. Movement of the shaft of the end effector 118 may also change the frequency relationship of the piezoelectric stack 122 to the end effector 118.

The piezoelectric stack 122 can be oriented such that expansions and contractions occur in a longitudinal direction, i.e. proximal and distal movement, and these movements are transmitted along the inner treatment probe 115. The piezoelectric stack 122 can cause movement of the end effector 118 in all directions (e.g., axial, side-to-side, etc.) The piezoelectric stack 122 can be oriented axially so the oscillation is applied in a longitudinal direction. The inner treatment probe 115 has a small outer diameter (e.g., 0.1 - 0.2 mm ) and can be flexible so that the axial motion of the stack 122 at its proximal end causes waves of vibration to be transmitted to the end effector 118 in all directions. In other words, the inner treatment probe 115 is designed to transmit movement of the piezoelectric stack 112 to the end effector 118 to cause vibrations of the end effector 118, but does not control the direction of movement of the end effector 118.

The piezo drive mechanism 120 can be used with a control board 121 which controls application of voltage to the piezoelectric crystal stack 122. The pattern and/or timing of the applied voltage can be varied to achieve different movements of the end effector 118, resulting in different vibrational frequencies and patterns. For example, varying a voltage in short, quick pulses can result in a higher vibration frequency than varying a voltage in longer, slower pulses.

A user can increase the frequency of vibrations in the end effector 118 for a more dramatic effect at the treatment location. For example, a greater magnitude or frequency of vibration at the end effector 118 can be selected if the user chooses to fully destroy the trabecular meshwork to allow for maximum outflow from the eye.

The piezoelectric crystals can be natural piezoelectric substrates, such as quartz single crystals, piezoelectric ceramics, such as lithium niobate, gallium arsenide, zinc oxide, aluminum nitride, or lead zirconate-titanate (PZT). In some implementations, the piezoelectric crystals are formed of polymer-film piezoelectrics, such as polyvinylidene fluoride. Such plastic-based crystal stacks can be lower cost and potentially disposable. The piezoelectric stack 122 can be a multilayer of thin piezoelectric/electrostrictive ceramic sheets stacked together. These multilayers have a relatively low driving voltage (100 V), quick response, high generative force, and high electromechanical coupling. The displacement of the piezoelectric crystals is sufficient to cause vibration of the end effector 118.

The device 100 can include a port or connection that is configured to receive tubing from a vacuum source to provide aspiration to the eye 5 during use, allowing evacuation of fluid and loosened particulate from the trabecular meshwork 10. The vacuum source can be an external or internal pump, including any of a variety of different aspiration pumps including volumetric flow or positive displacement pumps (e.g. peristaltic, linear peristaltic, piston, scroll pump) or vacuum-based pumps (e.g. venturi, pneumatic, diaphragm, or rotary-vane). The vacuum source can be located in, on, or otherwise near the outer housing 101 thereby minimizing a length of an aspiration line between the vacuum source and the distal tip of the elongate shaft 110 within the eye 5. Incorporating a vacuum source within the outer housing 101 minimizes the volume of the aspiration flow path improving control and responsiveness while decreasing latency or hysteresis. Vacuum can be applied to the lumen 116 of the treatment probe 115, the annular space between the inner surface of the treatment probe 115 and the outer surface of the core wire 117 (if core wire 117 is present), and/or the annular space between the inner surface of the outer sheath 113 and the outer surface of the treatment probe 115. Vacuum can be applied continuously or in discontinuous pulses during actuation of the piezo drive mechanism 120. As described in more detail below, the vacuum can be used in conjunction with irrigation to further enhance evacuation of freed particulate from the trabecular meshwork 10.

The device 100 can include a port or connection that is configured to receive tubing for supply of irrigation fluid to the eye 5 during use. The irrigation fluid, such as balanced saline solution (BSS), viscoelastic, or other irrigation fluids, can be supplied from an external source through tubing connected to device 100 such as via an irrigation sleeve (not shown). The irrigation sleeve can be positioned over the elongate shaft 110 to provide irrigation fluid from an irrigation line through one or more irrigation openings in the sleeve that are positioned within the eye 5 during use of the device 100. The irrigation fluid can also be coupled to the device 100 in a manner that allows the irrigation fluid to travel into the annular space between the external surface of the outer sheath 113 and the internal surface of the introducer tube 111. The irrigation fluid can also be coupled to the device 100 in a manner that allows the irrigation fluid to travel into the annular space between the external surface of the inner treatment probe 115 and the internal surface of the outer sheath 113. The irrigation fluid can also be coupled to the device 100 in a manner that allows the irrigation fluid to travel into the annular space between the inner surface of treatment probe 115 and the outer surface of core wire 117, and/or into the lumen 116 of treatment probe 115. The fluid can be delivered using passive hydrostatic pressure from an irrigation bag hung at a head height. The fluid can also be delivered using active pressure from a pump.

As seen in FIGS. 14A-14B, 15A-15B, and 16A-16B, the inner probe seal 145 fluidically isolates the space between the inner probe and the outer sheath 113 such that either vacuum or irrigation can be applied to the space. One or more ports for connection to line 144 can be located on the housing 101 distal of the inner probe seal 145, allowing vacuum or irrigation to be applied to the annular space between the outer sheath 113 and the inner treatment probe 115. Additionally, there can be a separator 146, which can house a sheath seal 147. A separator seal 148, such as an O-ring, can seal the outer diameter of the separator 146. The sheath seal 147 and separator seal 148 can fluidically isolate the annular space between the introducer tube 111 and the outer sheath 113 and create a separated space 149 within the housing 101 for vacuum or irrigation. The separator 146 can include a port which can fluidically connect the separated space 149 to a line 150 for irrigation and/or vacuum supply. The separated space 149 can then be used to apply irrigation and/or vacuum to the annular space between the introducer tube 111 and the outer sheath 113.

Power can be supplied to the device 100 such as via a cable 160 extending from a proximal end of the housing 101. The cable can also be configured to connect the device 100 to a wall socket. The device 100 can also be powered by one or more internal batteries. The battery can be incorporated within a region of the device 100, either internally or coupled to a region of the housing 101 such as within a modular, removable battery pack. The battery can have different chemical compositions or characteristics. For instance, batteries can include lead-acid, nickel cadmium, nickel metal hydride, silver-oxide, mercury oxide, lithium ion, lithium ion polymer, or other lithium chemistries. The device 100 can also include rechargeable batteries using either a DC power-port, induction, solar cells, or the like for recharging. Power systems known in the art for powering medical devices for use in the operating room are also to be considered herein such as spring power or any other suitable internal or external power source.

The device 100 can be designed to be a single-use, disposable device. Components of the device 100 can be formed of a metal and/or polymer material. For example, the housing 101, introducer tube 111, outer sheath 113, inner treatment probe 115, core wire 117, and end effector 118 can be formed of stainless steel, Nitinol, or plastic. The introducer tube 111 can be rigid, the outer sheath 113 and core wire 117 can be less rigid, and the inner treatment probe 115 and end effector 118 can be flexible, with respect to one another. The rigid introducer tube 111 can provide support for the outer sheath 113, core wire 117, and inner treatment probe 115. The outer sheath 113 and core wire 117 can be slightly less rigid, but still rigid enough to penetrate the trabecular meshwork 10 and rigid enough to hold the end effector 118 in the unexpanded configuration and/or compress the end effector 118 to expand it. The inner treatment probe 115 and end effector 118 can be flexible to aid in transmission of vibrations and/or if cannulation of Schlemm’s canal 12 is desired.

As an example method of use, the eye 5 can be penetrated by the distal end of the elongate shaft 110 of the device including at least a distal end of the introducer tube 111. An incision (e.g., 1.5 - 2.2 mm long) can be created using a cutting tool for clear corneal incisions or a puncture tool and the sulcus can be deepened using ophthalmic viscoelastic. The distal end region of the elongate shaft 110 can be inserted into and advanced through the anterior chamber 16 towards the target intraocular tissue such as the trabecular meshwork 10. There are various ways to approach the trabecular meshwork 10 and many techniques that can be employed depending on lens status, type and severity of the disease being treated.

Upon insertion of the introducer tube 111 into the eye 5, the outer sheath 113 can be inserted into the trabecular meshwork 10. The core wire 117, if present, can be advanced together with the outer sheath 113 and actuated, for example, by distally moving a secondary slider 132 through the trabecular meshwork 10. Outer sheath 113 can then be inserted more deeply by pushing the device distally and using core wire 117 as a guide, minimizing undesired tissue damage. Once the outer sheath 113 is at an appropriate depth, for example, so that the distal end of the outer sheath 113 is located within Schlemm’s canal, the end effector 118 can be expanded. In some implementations, the end effector 118 is actively expanded. The primary slider 131 can be retracted proximally to retract both the outer sheath 113 and the core wire 117. This exposes the the end effector 118 at the distal end region of the inner treatment probe 115 and also causes expansion of the end effector 118 towards the expanded state. Retraction of the core wire 117 axially compresses the end effector 118 to actively transition the end effector 118 to the expanded configuration, as described in detail above. Outer sheath retraction and core wire retraction can occur simultaneously upon actuation of the device with a single actuator. Alternatively, the outer sheath can retract first and the core wire retraction can occur second to expand the end effector 118. The end effector 118 of the inner treatment probe 115 can be positioned past the distal end of the outer sheath 113 by up to about 3 mm. Alternatively, the end effector 118 can be self-expanding and configured to automatically transition towards its expanded configuration upon being unsheathed from the outer sheath 113. In still further implementations, the end effector 118 need not expand and have the same outer diameter as the outer diameter of the adjacent regions of the treatment probe 115.

Once the end effector 118 has penetrated the trabecular meshwork 10 and is in its expanded configuration, the piezo drive mechanism 120 can be activated to cause vibration of the end effector 118. The vibrating end effector 118 can abut against one or more surrounding tissues to transmit forces against the tissue to thereby agitate particulates in the trabecular meshwork 10 and/or Schlemm’s canal. The agitation can loosen and free particulates from the treatment site to improve outflow from the anterior chamber. A trigger 140 can activate the piezo drive mechanism 120 and can be pressure-sensitive, with different pressure levels corresponding to different vibration frequencies, different amplitudes of vibration, or both. Alternatively, the pressure sensitive trigger 140 can activate the piezo drive mechanism 120 and control the vibration frequency with applied pressure, and a separate input can be used to control the vibration amplitude.

Irrigation and vacuum can be activated to evacuate fluids and/or particulate from the treatment site including from Schlemm’s canal and/or the trabecular meshwork 10. The inner treatment probe 115 and end effector 118 can be flexible to curve along a length of Schlemm’s canal 12 as discussed above. One or multiple insertions of probe 115 and effector 118 into Schlemm’s canal 12 can be performed in one or more locations along the canal to complete treatment. Once a section of Schlemm’s canal 12 and/or the trabecular meshwork 10 has been cleared of particulate, the insertion and agitation process can be repeated in another section.

Use of the terms “hand piece,” “hand-held,” or “handle” herein need not be limited to a surgeon’s hand and can include a hand piece coupled to a robotic arm or robotic system or other computer-assisted surgical system in which the user uses a computer console to manipulate the controls of the instrument. The computer can translate the user’s movements and actuation of the controls to be then carried out on the patient by the robotic arm.

The system can include a control unit, power source, microprocessor computer, and the like. Aspects of the subject matter described herein may be realized in digital electronic circuitry, integrated circuitry, specially designed ASICs (application specific integrated circuits), computer hardware, firmware, software, and/or combinations thereof. These various implementations may include an implementation in one or more computer programs that are executable and/or interpretable on a programmable system including at least one programmable processor, which may be special or general purpose, coupled to receive signals, data and instructions from, and to transmit signals, data, and instructions to, a storage system, at least one input device, and at least one output device.

These computer programs (also known as programs, software, software applications, or code) include machine instructions for a programmable processor, and may be implemented in a high-level procedural and/or object-oriented programming language, and/or in assembly/machine language. As used herein, the term “machine-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation”, “an implementation,” or the like, means that a particular feature, structure, configuration, or characteristic described is included in at least one embodiment or implementation. Thus, the appearance of the phrase “one embodiment,” “an embodiment,” “one implementation”, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/- 10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

What is claimed is:

1. An intraocular device for treating glaucoma, the device comprising:
a treatment probe comprising a distal end region and a proximal end region, the distal end region including a distal end effector sized to penetrate the trabecular meshwork;
an outer sheath surrounding at least the proximal end region of the treatment probe; and
a drive mechanism operatively coupled to a proximal end of the treatment probe and configured to cause oscillatory movement of the proximal end of the treatment probe to vibrate the distal end effector and agitate intraocular tissue in contact with the distal end effector.

2. The device of claim 1, wherein the drive mechanism further comprises a piezoelectric crystal stack.

3. The device of claim 2, wherein the piezoelectric crystal stack is driven at a frequency of 50 Hz - 40 kHz to vibrate the distal end effector.

4. The device of claim 3, wherein the distal end effector vibrates at a frequency that does not emulsify intraocular tissue.

5. The device of claim 1, wherein the device further comprises an input configured to control the drive mechanism.

6. The device of claim 1, wherein the distal end effector is configured to transition from an unexpanded state having a first outer diameter to an expanded state having a second outer diameter that is larger than the first outer diameter.

7. The device of claim 6, wherein the first outer diameter of the distal end effector in the unexpanded state is sized to be surrounded by the outer sheath.

8. The device of claim 7, wherein the distal end effector is self-expanding and transitions toward the expanded state upon exposure from the outer sheath.

9. The device of claim 8, wherein the device further comprises a core wire extending through a lumen of the treatment probe, and wherein the core wire is configured to transition the distal end effector from the unexpanded state to the expanded state.

10. The device of claim 1, wherein the device further comprises an irrigation channel for supplying irrigation fluid and a vacuum source for applying a vacuum through the device.

11. A method of treating glaucoma, the method comprising:
inserting a portion of an elongate shaft of an agitation device into the eye, the elongate shaft comprising:
a treatment probe including a distal end effector; and
an outer sheath having a distal end region surrounding the distal end effector of the treatment probe; and
penetrating a trabecular meshwork with the distal end region of the outer sheath;
exposing the distal end effector from the outer sheath; and
vibrating the distal end effector within Schlemm’s canal to agitate intraocular tissue in contact with the distal end effector.

12. The method of claim 11, wherein vibrating the distal end effector to agitate intraocular tissue loosens particulate from the trabecular meshwork.

13. The method of claim 11, wherein vibrating the distal end effector comprises actuating a drive mechanism operatively coupled to a proximal end of the treatment probe to cause oscillatory movement of the proximal end of the treatment probe.

14. The method of claim 13, wherein the drive mechanism further comprises a piezoelectric crystal stack.

15. The method of claim 14, wherein the piezoelectric crystal stack is driven at a frequency of 50 Hz - 40 kHz to vibrate the distal end effector.

16. The method of claim 15, wherein the distal end effector vibrates at a frequency that does not emulsify intraocular tissue.

17. The method of claim 11, wherein exposing the distal end effector from the outer sheath comprises transitioning the distal end effector from an unexpanded state having a first outer diameter to an expanded state having a second outer diameter that is larger than the first outer diameter.

18. The method of claim 17, wherein the distal end effector is self-expanding and transitions toward the expanded state upon proximal withdrawal of the outer sheath from the distal end effector.

19. The method of claim 17, wherein the distal end effector is actively expanded and transitions toward the expanded state upon proximal withdrawal of the outer sheath from the distal end effector and proximal retraction of a core wire extending through a lumen of the treatment probe to axially compress the distal end effector.

20. The method of claim 11, further comprising supplying irrigation fluid through the elongate shaft; and applying vacuum through the elongate shaft.
